(19) United States
(12) Patent Application Publication
(10) Pub. No.: US 2008/0171930 A1
(43) Pub. Date: Jul. 17, 2008

(54) METHOD AND APPARATUS FOR POSITIONING AN INSTRUMENT IN A PREDETERMINED REGION WITHIN A PATIENT'S BODY

(75) Inventors: Amir Abolfathi, Woodside, CA (US); Mahmood K. Razavi, Irvine, CA (US)

Correspondence Address: LEVINE BAGADE HAN LLP, 2483 EAST BAYSHORE ROAD, SUITE 100, PALO ALTO, CA, 94303, US

(73) Assignee: AR2 Partners, Inc., Woodside, CA (US)

(21) Appl. No.: 11/623,713

(22) Filed: Jan. 16, 2007

(51) Int. Cl. A61B 17/00

(52) U.S. Cl. 600/410; 606/1

(57) ABSTRACT

A method and apparatus for positioning an instrument in a predetermined region within a patient's body is disclosed herein generally having a custom fitted surgical guide template which corresponds to a digital model of the portion of the patient's body. The custom fitted surgical guide template is positionable about a portion of the patient's body surface that is identifiable and unique for each patient which is fixed relative to adjacent anatomical structures. The custom fitted surgical guide may also have at least one instrument guiding mechanism which is positioned to guide an instrument along a predetermined trajectory into the predetermined region within the patient's body. The instrument may be advanced through the instrument guiding mechanism using a steering mechanism regulated by a physiological parameter.

### BACKGROUND OF THE INVENTION

The present invention relates to apparatus and methods for performing surgery or other procedures on a portion of a patient's body. More particularly, the present invention relates to apparatus and methods for locating and accessing an area of interest, such as a tumor or lesion, using a custom fitted surgical guide template manufactured from a three dimensional computerized model gathered from an imaging system (e.g. CT, MRI, ultrasound, etc.).

Surgery or procedure may be performed to investigate, implant, diagnose, repair, or remove anomalies located within a portion of a patient's body. However, each time a device, such as a needle or an electrode, is passed through the brain, there is a risk of intracranial hemorrhaging. As a result, stereotactic surgery was introduced to assist with locating an area of interest within the brain. Currently, stereotactic surgery involves the registration of medical images, typically from an MRI and/or CT, with a surgical coordinate system. For most operations, this coordinate system is defined by a set of x, y, and z marks on a surgical frame which is affixed to a patient's skull. The surgical frame is usually fixed to the patient's skull via screws and can weigh several pounds. The frame may be quite cumbersome and uncomfortable for the patient as well as exposing the patient to a risk of infection at the insertion sites. In addition, operation room time may be wasted making multiple mechanical adjustments to the surgical frame.

Accordingly, there is a need for apparatus and methods that decrease operating room time and over-all effectiveness of the procedure by providing a custom fitted surgical guide that is custom fitted for each patient for easy and safe access to an area of interest within the anatomy.

### BRIEF SUMMARY OF THE INVENTION

An apparatus and method for positioning an instrument in a predetermined region within a patient's body, such as the skull (brain), spine, torso (lungs, liver, heart, kidneys), joints, nodes, muscles, and/or soft tissue, may generally comprise, in one variation a custom fitted surgical guide template which correlates to a digital model. The custom fitted surgical guide template may further comprise of at least one mechanism to guide the instrument along a computed pathway which corresponds to a predetermined trajectory.

The digital model may be generated by scanning the portion of the patient's body using an imaging system, such as a magnetic resonance imaging (MRI), a computed tomography (CT) system, and/or ultrasound, and compiling the data using a computer. A region to be treated may be identified and the pathway from outside the patient's body to the region to be treated may be computed using the digital model. Computing the pathway may comprise assigning x and y coordinates to the region to be treated relative to an assigned datum whereby the x and y coordinates determine the position and trajectory of the instrument guiding mechanism. Computing the pathway may further comprise assigning a z coordinate to the region to be treated whereby the z coordinate determines the depth of the instrument relative to the region to be treated.

The custom fitted surgical guide template may be manufactured such that it correlates to the digital model and has at least one instrument guiding mechanism pre-positioned to guide the instrument along the computed pathway which corresponds to the predetermined trajectory. The custom fitted surgical guide template is designed and fabricated in a manner that the contact surfaces of the custom fitted guide can only be positioned over a pre determined anatomical region that is identifiable and unique for each patient. Once the custom fitted surgical guide is placed on the patient body, there is fixed or limited in movement relative to an adjacent anatomical structure upon which the template is positioned. The custom fitted surgical guide template may be in the shape of a frame or scaffold and/or alternatively a helmet or covering sized to fit at least partially over a head of a patient or other region of the patient's body. The custom fitted surgical guide template may be comprised of a variety of materials such as polymers, plastic and/or alternatively metal. In addition, the custom fitted surgical guide template may also comprise a mechanism to position the custom fitted surgical guide template about the portion of the patient's body which is fixed relative to adjacent anatomical structures.

Additionally, the instrument guiding mechanism may be a port or alternatively, a plurality of ports, which are positioned at a predetermined angle relative to the custom fitted surgical guide template. The port may stationary and/or coupled to the custom fitted surgical guide template such that he angle may be adjusted by applying pressure to the port. However, the port may be secured in place by a locking mechanism. The instrument may be positioned in a predetermined region within a patient's body by advancing the instrument along at least one instrument guiding mechanism. The instrument may also be minimally invasive or noninvasive and may be positioned adjacent to a predetermined area of a patient's body.

Furthermore, the instrument may comprise an indicator therealong located at a predefined location that when compared to the custom fitted surgical guide template is indicative of a depth of the instrument relative to the predetermined region to be treated within the patient body. The indicator may be, in one variation, an indentation or groove which is sized to engage a complementary protrusion along the custom fitted surgical guide template such that engagement of the protrusion within the indentation or groove stops advancement of the instrument into the patient's body. The protrusion may comprise at least one spring or biasing element which urges the protrusion into contact against the surgical instrument. In another variation, the indicator may be at least one protrusion whereby the advancement of the instrument within the patient's body is stopped when the protrusion is proximal to the entry port. In yet another variation, the indicator may be one or more visual markings.

### BRIEF DESCRIPTION OF THE SEVERAL VIEWS OF THE DRAWINGS

FIG. 1 schematically illustrates exposing a patient to an imaging system whereby data is collected from the imaging system to a computer.

FIG. 2 schematically illustrates scan slices of a portion of a patient's brain compiled by the computer in three dimensions.

FIG. 3 schematically illustrates a three dimensional model of a portion of a patient's brain.

FIG. 4 schematically illustrates a three dimensional model of a portion of a patient's head.

FIGS. 5A-5B illustrate perspective and front views, respectively, of a custom fitted surgical guide template with a mechanism for inserting a surgical instrument at a pre-defined angle.

FIG. 6 shows a variation of the custom fitted surgical guide template as shown in FIG. 5B in the shape of a frame or scaffold.

FIG. 7 shows a variation of the custom fitted surgical guide template as shown in FIG. 6.

FIG. 8 illustrates a variation of the custom fitted surgical guide template as shown in FIG. 5B where the mechanism for guiding an instrument is a port.

FIG. 9 shows a variation of the custom fitted surgical guide template as shown in FIG. 8.

FIG. 10 shows a variation of the custom fitted surgical guide template as shown in FIG. 8 where the port is flexible and may move in a 360° direction.

FIG. 11 shows a variation of the custom fitted surgical guide template as shown in FIG. 8 where a proximal end of the port relative to the patient's body is coupled to the custom fitted surgical guide template in a ball and socket type configuration to allow the port to be moved in a 360° direction.

FIG. 12 shows a variation of the custom fitted surgical guide template as shown in FIG. 11 where the port is held in a predetermined position by springs.

FIG. 13 shows a variation of the custom fitted surgical guide template as shown in FIG. 11 where the port is locked in a predetermined position by a locking mechanism.

FIG. 14 shows a variation of the custom fitted surgical guide template as shown in FIG. 8 containing more than one port.

FIGS. 15A-15B show front and perspective views, respectively, of a custom fitted surgical guide template shaped like a helmet and containing straps.

FIG. 16 shows the custom fitted surgical guide template as illustrated in FIGS. 15A-15B positioned on a patient's head secured via straps.

FIG. 17 shows a variation of the custom fitted surgical guide template as shown in FIG. 16 positioned on a patient's head secured via screws or pins.

FIG. 18 shows another variation of a custom fitted surgical guide template as shown in FIG. 5B containing a non-slip surface positionable again the patient's head.

FIG. 19 shows a variation of the custom fitted surgical guide template as shown in FIG. 18.

FIG. 20 schematically illustrates a custom fitted surgical guide template positioned on a patient's head via straps with a surgical instrument inserted through a port on the custom fitted surgical guide template to a target location.

FIG. 21 shows a custom fitted surgical instrument to be inserted into a port to a depth indicated on the surgical instrument.

FIG. 22A-22B illustrates a cross-sectional view of a port with a protrusion and a surgical instrument with an indentation such that engagement of the protrusion within the indentation stops the instrument at a predetermined depth, respectively.

FIG. 23 shows a surgical instrument to be inserted into a port to a depth indicated by a protrusion along a length of the surgical instrument.

FIG. 24 illustrates a variation of the surgical instrument which contains a protrusion or a stopper.

FIG. 25 schematically illustrates a lead placed at a target location using the custom fitted surgical guide template.

FIG. 26 schematically illustrates an instrument that is minimally invasive or noninvasive that may transmit and/or guide energy using the custom fitted surgical guide template.

FIG. 27 illustrates another variation of a custom fitted surgical guide template positioned around a patient's knee.

FIG. 28 illustrates a variation of a custom fitted surgical guide template positioned around a portion of a patient's torso.

FIG. 29 schematically illustrates a biofeedback unit in communication with a controller unit which guides an instrument through a port using a steering mechanism.

FIG. 30 illustrates an exploded side view of the steering mechanism within the port as shown in FIG. 29.

### DETAILED DESCRIPTION OF THE INVENTION

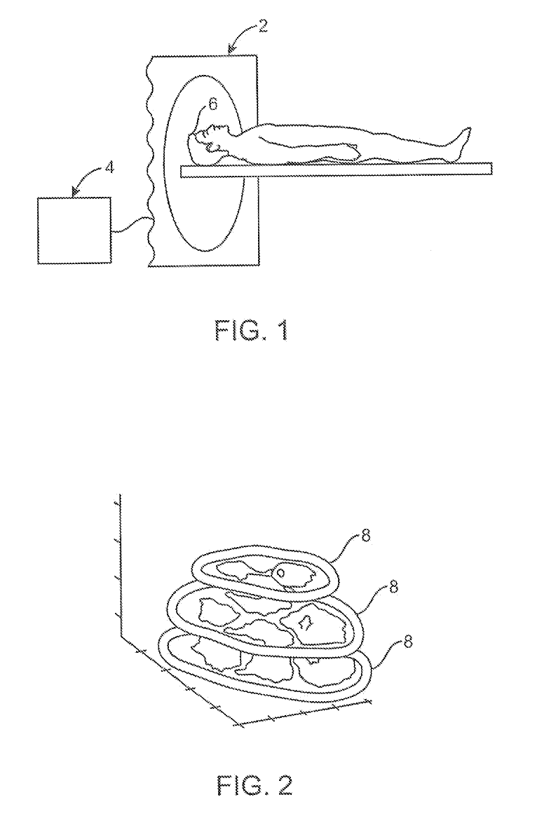

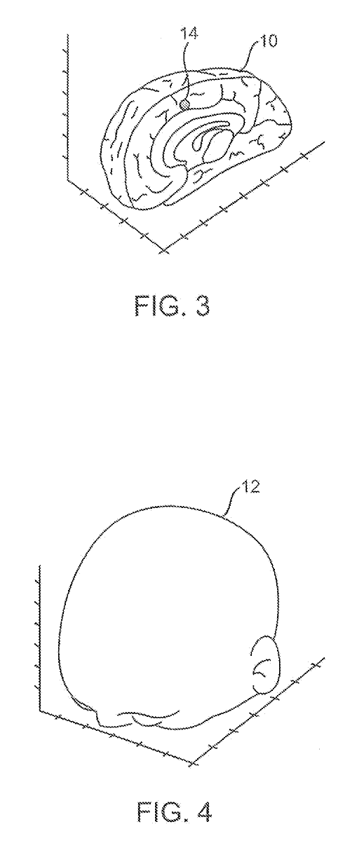

Medical imaging systems 2, such as magnetic resonance imaging (MRI), computed tomography (CT) and ultrasound (US) in communication with a computer 4 provide an avenue for physicians to view a region of interest within a patient's body. As shown in FIG. 1, a portion of the patient's body, such as the head 6, may be scanned by an imaging system 2. The scan typically produces a series of slices or two dimensional images 8. The computer 4 may construct a composite of the slices 8 to generate a digital model of the region of interest 10 and/or the portion of the patient's body 12 which may be superimposed on a three dimensional grid, as demonstrated in FIGS. 2-4. Additionally, the computer 4 may provide a direct computation of the x, y, and z coordinate values of any location of interest 14 within the digital model 10 on the grid. Accordingly, the physician may study the digital images, either the two dimensional scanned slices 8 and/or the three dimensional digital model 10 to determine the x, y, and z coordinates of the target 14 within a region of interest. In addition to identifying the target 14, it is often critical for the physician to also determine a suitable pathway from outside the patient's body through the tissue and to the target 14 in order to minimize damage to the surrounding tissue. Thus, identifying the safest pathway to the target 14 which damages the least amount of tissue is very desirable.

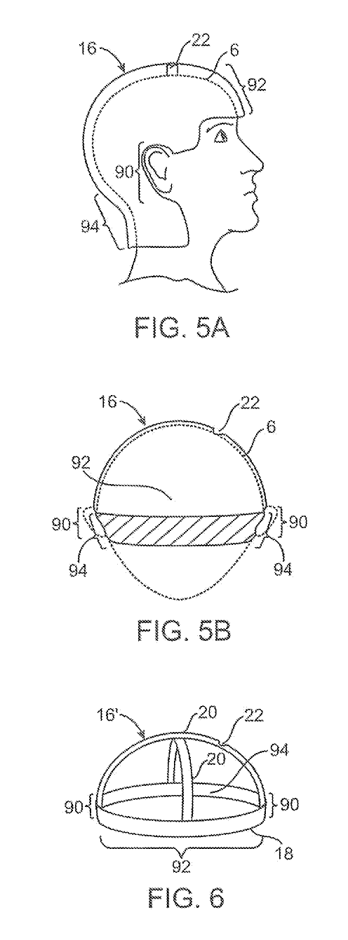

Once the target 14 and the pathway are identified, a custom fitted surgical guide template 16 for positioning an instrument 34 in a predetermined region within a patient's body may be manufactured prior to surgery. The custom fitted surgical guide template 16 may be fabricated to correspond to the size and shape of the digital model of the portion of the patient's body 12. Moreover, the custom fitted surgical guide template 16 may be positioned over various regions of the patient body so long as the region of the body to be treated is fixed or limited in movement relative to anatomical surface structure and contour upon which the template 16 is positioned. The custom fitted surgical guide template 16 may be made of a rigid material that is common in the art, such as polymers (permanent and/or at least partially absorbable), plastic and/or metal. As illustrated in FIGS. 5A-5B, the custom fitted surgical guide template 16 may be in the shape of a helmet or covering which fits at least partially over the head 6 of a patient. The surgical guide template 16 may be contoured to correspond to the patient's unique anatomical features, including by example but not limited to the location of ears 90 and/or shape of the forehead 92 and/or neck 94 or any other distinguishing anatomical feature unique to the patient. The contours of such anatomical features as well as the relative positioning and location between these features may be integrated into the surgical guide template 16 such that the template 16 is custom-fitted not only to a specific patient but is also custom-fitted to align specifically with these features in a pre-determined manner. The thickness of the custom fitted surgical guide template 16 from the surface positionable against the patient's body relative to the surface opposite may vary depending on the material and structure. The custom fitted surgical guide template 16 should be thick enough to maintain rigidity. For example, the thickness may be between 0.2 cm and 1.0 cm, although the thickness may be more or less.

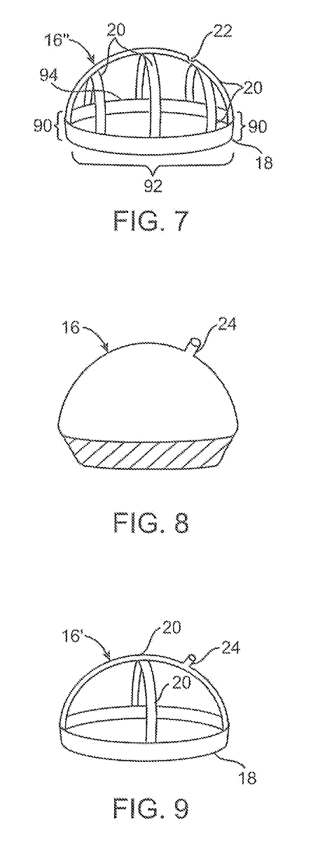

Alternatively, as illustrated in FIGS. 6-7, the custom fitted surgical guide template may be a frame or scaffold 16′ and 16″. The frame or scaffold 16′ and 16″ may contain a first structure 18 that covers at least a portion of the patient's body surface with at least one second structure 20 that covers at least a portion of the patient's body surface at an angle relative to the first structure 18. The width of the first 18 and second structures 20 should be wide enough to also maintain rigidity. For example, the width of the first 18 and second structures 20 may be between 1 cm and 3 cm, although the width may be more or less or in any combination. Although these examples show use for neurosurgical applications, the custom fitted surgical guide template may be configured to treat various other parts of the body.

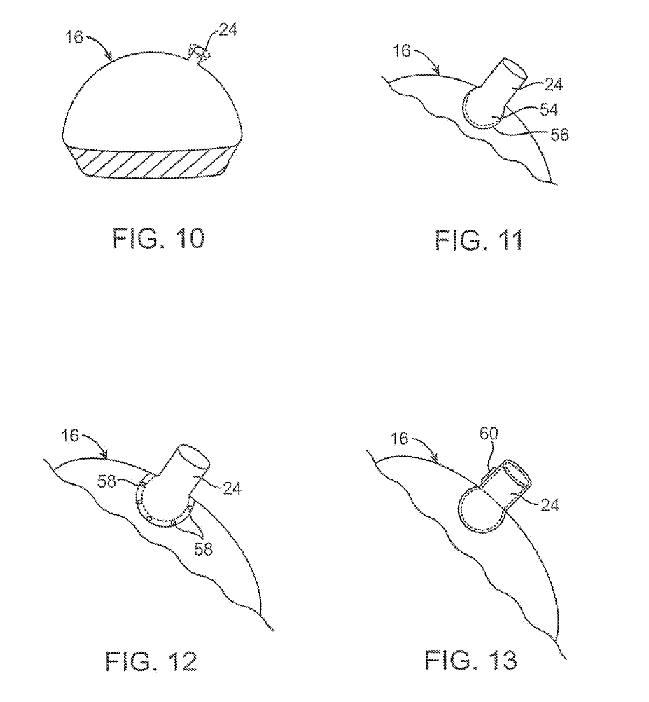

The custom fitted surgical guide template 16 may contain at least one instrument guiding mechanism 22 which is positioned to guide an instrument 34 (e.g. needle, probe, electrode, transducer, etc.) along a predetermined trajectory into a predetermined region within the patient's body. The location on the custom fitted surgical guide template 16 and the trajectory of the instrument 34 may be determined by the pathway computed and the x and y coordinates assigned previously be the physician. As shown in FIGS. 8-9, the instrument guiding mechanism may be an entry port 24 having a predetermined angle relative to the custom fitted surgical guide. The predetermined angle corresponds to the trajectory of the instrument 34. The port 24 may be used to preserve the pathway as well as guide instruments 34 along the desired pathway. However, the predetermined angle may be adjusted during the procedure by the physician to allow the instrument 34 to follow an alternate pathway to the predetermined region and/or to reach an area outside the predetermined region. In one variation, as shown in FIG. 10, the angle may be adjusted by applying pressure to the port 24 where the port 24 is made of a flexible material (such as a polymer). In another variation, the angle may be adjusted by rotating the proximal end of the port 24 relative to the patient's body within the custom fitted surgical guide template 16. As shown in FIG. 11, the proximal end of the port 24 may be coupled with the custom fitted surgical guide template 16 in a ball 54 and socket 56 configuration. Once the port 24 is moved, it may be secured at the adjusted angle through pressure from the ball 54 and socket 56 contact. In yet another variation, as illustrated in FIG. 12, the port 24 may be held in the predetermined angle by at least one spring 58. The angle may be adjusted by applying pressure to the port 24 and/or springs 58. As shown in FIG. 13, the port 24 may be secured in place by a removable locking mechanism 60 to prevent the port 24 from being adjusted from the predetermined angle.

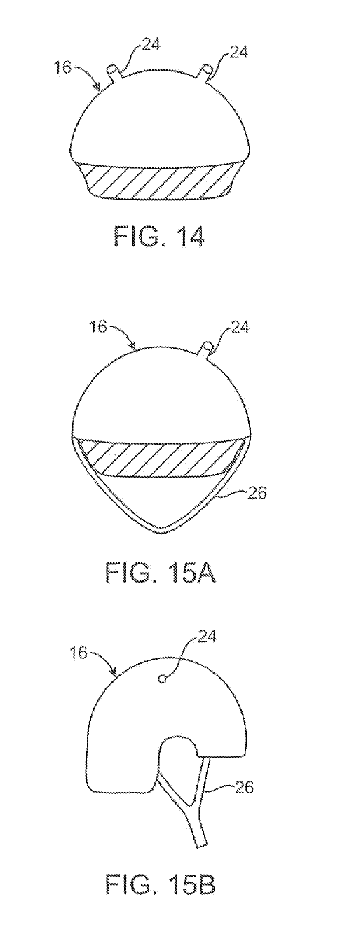

Furthermore, as shown in FIG. 14, a plurality of ports may be present on the custom fitted surgical guide template 16. The ports 24 may be at different angles relative to other ports on the custom fitted surgical guide template 16 and may direct the one or more instrument 34 to the target location 14 through alternative pathways and/or may urge the instrument 34 to more than one target location.

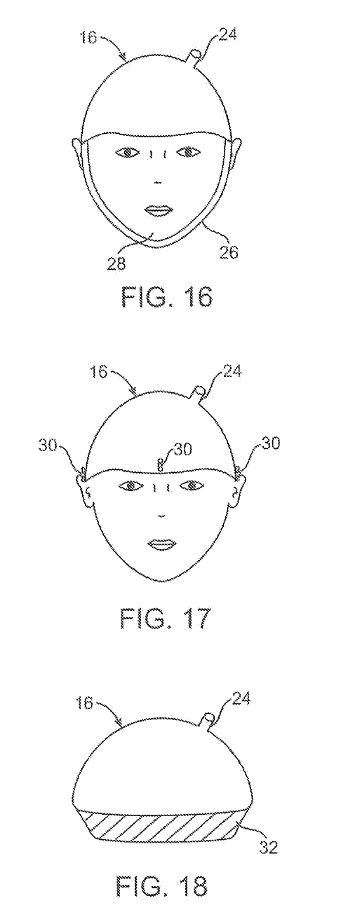

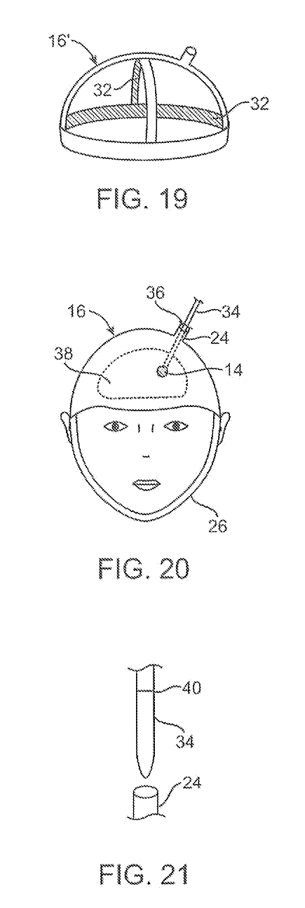

The custom fitted surgical guide template 16 is positioned about a pre determined portion of the patient's body surface and then may be fixed relative to adjacent anatomical structures via any number of mechanisms to prevent it from mobilization during the procedure. For example, FIG. 15A-15B shows a variation of the custom fitted surgical guide template 16 containing at least one strap 26. As shown in FIG. 16, the custom fitted surgical guide template 16 may be positioned on a patient's head 6 and may be fixed to the head 6 by securing at least one strap about the patient's chin 28. The strap 26 may be secured about other areas of the body such as the neck, shoulders, or arms, etc. Another variation may include at least one screw 30 or pin affixable or secured at least temporarily to the patient's head, as shown in FIG. 17. In yet another variation, the custom fitted surgical guide template 16 may comprise a non-slip surface 32 positionable against the patient's body, as shown in FIGS. 18-19. The non-slip surface 32 may be filled, coated, layered and/or otherwise made with and/or from a material known to one having ordinary skill in the art to fix to a portion of a patient's body. For example, the non-slip material may include rubber or surgical adhesive, etc.

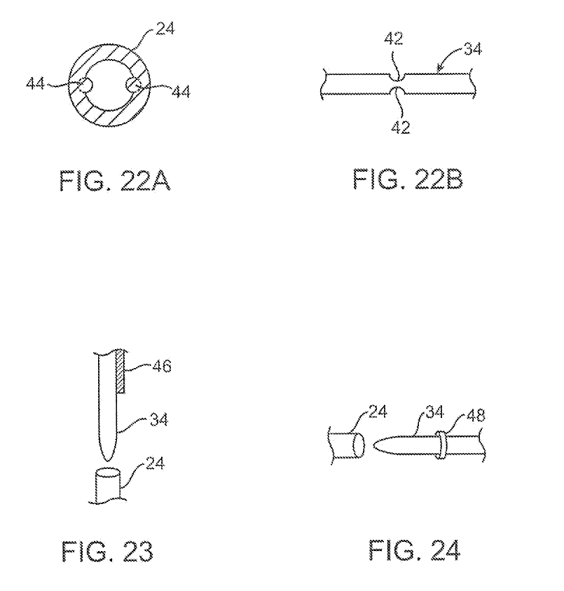

As demonstrated in FIG. 20, once the custom fitted surgical guide template 16 is positioned and fixed to a portion of the patient's body, an instrument 34 may be advanced along the instrument guiding mechanism 22 and/or port 24 into the patient's body to the target location 14 within the predetermined region of interest. In order to reach the target location 14, the instrument 34 may have an indicator 36 located at a predefined location that when compared to the custom fitted surgical guide template 16 and/or port 24 is indicative of a depth of the instrument 34 relative to the predetermined region 38. The predefined location may be determined by the pathway computed and z coordinate assigned previously be the physician so that the advancement of the instrument 34 is stopped at the target location 14. FIGS. 21-24 are some examples of the indicator 36. FIG. 21 illustrates one variation where the indicator is visual, such as a line or marking 40 on the instrument 34 where the advancement of the instrument 34 is stopped when the marker 40 is adjacent to the port 24. FIGS. 22A-22B illustrate another variation where the indicator is an indentation or groove 42 on the instrument 34 which is sized to engage a complementary protrusion 44 along the custom fitted surgical guide template 16 and/or port 24. The protrusion 44 may include at least one spring or biasing element which urges the protrusion 44 into contact against the indentation 42 on the instrument 34. When the protrusion 44 engages with the indentation 42 on the instrument 34, the advancement of the instrument 34 into the patient's body is stopped. FIGS. 23-24 illustrate yet another variation where the indicator comprises at least one protrusion where the advancement of the instrument 34 is stopped when the protrusion is proximal to the port 24. FIG. 23 shows a protrusion along a length 46 of the instrument 34. FIG. 24 shows a protrusion around a portion 48 of the instrument 34. However, any protrusion that may stop the advancement of the instrument 34 may be used.

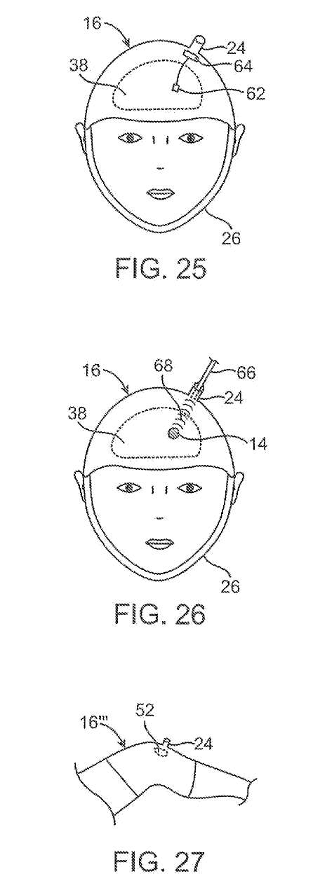

When the instrument 34 has reached the target location 14, it may be used to diagnose, treat, monitor, etc. the region of interest within a patient's body. Some non-limiting examples include placing sensors or leads 62 in the region of interest connected to a neurostimulator 64, as shown in FIG. 25. The instrument 34 may also be used for delivering implants, energy, or drugs. The instrument 34 may additionally be used to remove and/or treat tumors, lesions, fibroids, stones, etc. For example, FIG. 26 illustrates a minimally invasive or noninvasive instrument, such as a transducer 66, which may be advanced along the instrument guiding mechanism 22 and/or port 24 to the patient's body at a predetermined angle and trajectory such that focused energy 68 (e.g. ultrasound, laser, radiofrequency, microwave, etc.) may be guided to the target location 14.

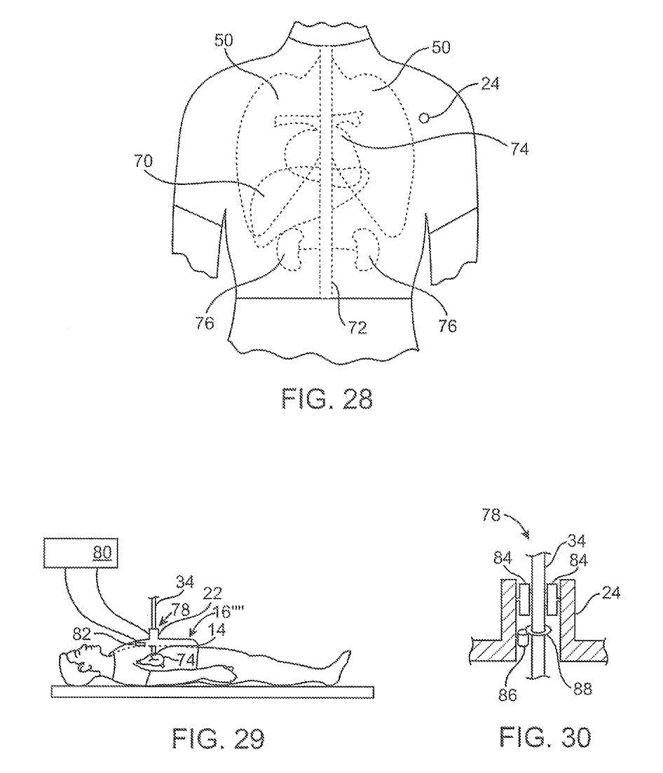

In addition to using the custom fitted surgical guide template on the patient's head, the custom fitted surgical guide template may be positioned on other portions of the body that correspond to regions that are fixed relative to the exterior of the body, such as the spine 72, lungs 50, liver 70, heart 74, kidneys 76, joints 52, nodes, muscles, soft tissue, etc. using the same method as described above for the brain. FIGS. 27-28 show alternative portions of the body. FIG. 27 illustrates a custom fitted surgical guide template 16′″ positioned about a patient's knee to advance an instrument 34 through the port 24 into the joint 52. In addition to the joints 52, a custom fitted surgical guide template 16″″ may be positioned about a patient's torso to advance an instrument 34 through the instrument guiding mechanism 22 into the chest to a region of interest, such as the lungs 50 or heart 74, as shown in FIG. 28.

As shown in FIGS. 29-30, the custom fitted surgical guide template 16″″ may also contain a steering mechanism 78 which may be used to advance the instrument 34 through the instrument guiding mechanism 22 to the predetermined target location 14. The steering mechanism 78 may contain at least one servomotor and may be at least partially located within the custom fitted surgical guide template 16″″. However, the steering mechanism 78 may be external to the custom fitted surgical guide template 16″″. The servomotor may be regulated by a controller unit 80 according to a physiological parameter of the patient's body. The physiological parameter may be measured using a biofeedback unit 82 in wired or wireless communication with the controller unit 80. The biofeedback unit 82 may be worn directly on the user's body or it may be held in proximity to the body to detect any number of physiological parameters, including, but not limited to, heartbeat, blood pressure, respiration, and/or cardiac cycle, etc. FIG. 29 illustrates a biofeedback unit 82 secured to the chest of a patient such that a heartbeat may be detected and communicated to the controller unit 80. However, the biofeedback unit 82 may be securable to any portion of the body which is able to provide a physiological parameter. The biofeedback unit 82 may be in communication with the controller unit 80 via a wired connection or it may be configured to communicate wirelessly. Accordingly, the biofeedback unit 82 may be equipped with a transmitter and the controller unit 80 may be equipped with a receiver adapted to receive a wireless signal from the biofeedback unit 82, e.g. via infrared.

The controller unit 80 may regulate the advancement of the instrument 34 such that the instrument 34 is guided along the predetermined trajectory between, during, and/or at a particular stage of a heartbeat and/or respiration. The instrument 34 may be advanced via a steering mechanism 78 containing at least one servomotor. As shown in FIG. 30, the steering mechanism 78 may contain a first servomotor 84 which may be used for linear movement. The steering mechanism 78 may also contain a second servomotor 86 which may be used for angular movement. The second servomotor 86 may contain an arm or joint 88 which may be used to stabilize the instrument 34.

While illustrative examples are described above, it will be apparent to one skilled in the art that various changes and modifications may be made therein. Moreover, various apparatus or methods described above are also intended to be utilized in combination with one another, as practicable. The appended claims are intended to cover all such changes and modifications that fall within the true spirit and scope of the invention.

What is claimed is:

1. An apparatus for positioning an instrument in a predetermined region within a patient's body, comprising:
a custom fitted surgical guide template positionable about a portion of the patient's body surface which is fixed relative to adjacent anatomical structures, wherein the custom fitted surgical guide has at least one instrument guiding mechanism which is positioned to guide an instrument along a predetermined trajectory into the predetermined region within the patient's body, and
wherein the custom fitted surgical guide template corresponds to a digital model of the portion of the patient's body.

2. The apparatus of claim 1, wherein the instrument is minimally invasive or noninvasive.

3. The apparatus of claim 1, wherein the predetermined region is an area within the patient's body selected from the group consisting of a brain, joint, lung, liver, spine, heart, kidney, node, muscle, and soft tissue.

4. The apparatus of claim 1, wherein the custom fitted surgical guide template comprises a helmet sized to fit at least partially over a head contour of a patient.

5. The apparatus of claim 1, wherein the custom fitted surgical guide template comprises a frame or scaffold.

6. The apparatus of claim 1, wherein the custom fitted surgical guide template is comprised of a polymer.

7. The apparatus of claim 1, wherein the custom fitted surgical guide template is comprised of a plastic.

8. The apparatus of claim 1, wherein the custom fitted surgical guide template is comprised of a metal.

9. The apparatus of claim 1, wherein the custom fitted surgical guide template is positionable and then immobilized about the portion of the patient's body via at least one strap.

10. The apparatus of claim 1, wherein the custom fitted surgical guide template is positionable about the portion of the patient's body and then immobilized via at least one screw or pin affixable at least temporarily to the patient's body.

11. The apparatus of claim 1, wherein the custom fitted surgical guide template further comprises a non-slip surface positionable against the patient's body.

12. The apparatus of claim 11, wherein the non-slip surface is comprised of rubber.

13. The apparatus of claim 1, wherein the instrument guiding mechanism comprises a port having a predetermined angle relative to the custom fitted surgical guide template.

14. The apparatus of claim 13, wherein the predetermined angle may be adjusted by applying pressure to the port.

15. The apparatus of claim 14, wherein the port is coupled to the custom fitted surgical guide template in a ball and socket configuration and is rotateable relative to the custom fitted surgical guide template.

16. The apparatus of claim 14, wherein the port is coupled to the custom fitted surgical guide template using at least one spring.

17. The apparatus of claim 14, wherein the port is secured in place by a locking mechanism.

18. The apparatus of claim 13, further comprising a plurality of ports.

19. The apparatus of claim 13, wherein the instrument comprises an indicator therealong located at a predefined location that when compared to the custom fitted surgical guide template is indicative of a depth of the instrument relative to the predetermined region.

20. The apparatus of claim 19, wherein the indicator comprises an indentation or groove which is sized to engage a complementary protrusion along the custom fitted surgical guide template such that engagement of the protrusion within the indentation or groove stops advancement of the instrument into the patient's body.

21. The apparatus of claim 20, wherein the protrusion comprises at least one spring or biasing element which urges the protrusion into contact against the surgical instrument.

22. The apparatus of claim 19, wherein the indicator comprises at least one protrusion whereby the advancement of the instrument within the patient's body is stopped when the protrusion is proximal to the entry port.

23. The apparatus of claim 19, wherein the indicator comprises a visual marking.

24. The apparatus of claim 1, further comprising an imaging system for generating the digital model.

25. The apparatus of claim 24, wherein the imaging system is a magnetic resonance imaging, ultrasound, or computed tomography system.

26. The apparatus of claim 1, wherein the instrument is guided by a steering mechanism.

27. The apparatus of claim 26, wherein the steering mechanism comprises at least one servomotor.

28. The apparatus of claim 26, further comprising:
a controller unit in communication with the steering mechanism; and
a biofeedback unit adapted to detect a physiological parameter from the patient's body and which is in communication with the controller unit.

29. The apparatus of claim 28, wherein the biofeedback unit is adapted to detect a heartbeat.

30. The apparatus of claim 28, wherein the biofeedback unit is adapted to detect respiration.

31. The apparatus of claim 28, wherein the controller unit is adapted to regulate the steering mechanism such that the instrument is guided along the predetermined trajectory between, during, and/or at a particular stage of a heartbeat and/or respiration.

32. A method for positioning an instrument in a predetermined region within a patient's body, comprising:
positioning a custom fitted surgical guide template about a portion of the patient's body surface which is fixed relative to adjacent anatomical structures; and
advancing a surgical instrument along at least one instrument guiding mechanism which is positioned on the custom fitted surgical guide template to guide the instrument along a predetermined trajectory into the patient's body to a predetermined region to be treated, wherein the custom fitted surgical guide template corresponds to a digital model of the portion of the patient's body.

33. The method of claim 32, wherein positioning comprises positioning the custom fitted surgical guide template via at least one strap.

34. The method of claim 32, wherein positioning comprises positioning the custom fitted surgical guide template via at least one screw or pin affixable at least temporarily to the patient's body.

35. The method of claim 32, further comprising scanning the portion of the patient's body which is fixed relative to adjacent anatomical structures with an imaging system to obtain digital data representative of the portion prior to positioning.

36. The method of claim 35, wherein the imaging system is a magnetic resonance imaging, ultrasound, or computed tomography system.

37. The method of claim 36, further comprising processing the data with a computer to generate a three dimensional model of the portion.

38. The method of claim 32, further comprising identifying the region to be treated within the three dimensional model.

39. The method of claim 38, further comprising computing a pathway from outside the patient's body to the region to be treated.

40. The method of claim 39 wherein computing a pathway comprises assigning x and y coordinates to the region to be treated whereby the x and y coordinates determine the position and trajectory of the instrument guiding mechanism.

41. The method of claim 40, further comprising assigning a z coordinate to the region to be treated whereby the z coordinate determines the depth of the instrument relative to the region to be treated.

42. The method of claim 39, further comprising manufacturing the custom fitted surgical guide template such that it correlates to the three dimensional model, wherein the custom fitted surgical guide template comprises at least one entry port pre-positioned to guide the instrument along the computed pathway which corresponds to the predetermined trajectory.

43. The method of claim 42, wherein the predetermined trajectory may be adjusted by applying pressure to the port.

44. The method of claim 32, wherein advancing comprises guiding the instrument to an area within a patient's body selected from the group consisting of a brain, joint, lung, liver, spine, heart, kidney, node, muscle, and soft tissue.

45. The method of claim 32, wherein advancing comprises inserting the instrument along the instrument guiding mechanism to the region to be treated wherein the instrument comprises an indicator therealong located at a predefined location that when compared to the custom fitted surgical guide template is indicative of a depth of the instrument relative to the predetermined region.

46. The method of claim 45, wherein the indicator comprises an indentation or groove which is sized to engage a complementary protrusion along the custom fitted surgical guide template such that engagement of the protrusion within the indentation or groove stops advancement of the instrument into the patient's body.

47. The method of claim 46, wherein the protrusion comprises at least one spring or biasing element which urges the protrusion into contact against the surgical instrument.

48. The method of claim 45, wherein the indicator comprises at least one protrusion whereby the advancement of the instrument within the patient's body is stopped when the protrusion is proximal to the entry port.

49. The method of claim 45, wherein the indicator comprises a visual marking.

50. The method of claim 32, wherein advancing the instrument comprises advancing the instrument using a steering mechanism.

51. The method of claim 50, further comprising measuring a physiological parameter of the patient's body.

52. The method of claim 51, wherein measuring a physiological parameter comprises detecting a heartbeat and/or respiration.

53. The method of claim 52, wherein the physiological parameter is detected via a biofeedback unit.

54. The method of claim 52, further comprising regulating the advancement of the instrument via a controller unit between, during, and/or at a particular stage of a heartbeat and/or respiration.
